(19) United States
(12) Patent Application Publication
(10) Pub. No.: US 2007/0237773 A1
(43) Pub. Date: Oct. 11, 2007

(54) HUMAN SEMA4A PROTEIN ANTIBODIES AND USES THEREOF

(76) Inventors: Hitoshi KIKUTANI, Suita Osaka (JP); Atsushi Kumanogoh, Ikeda Osaka (JP); Edward Leon Barsoumian, Toyonaka Osaka (JP)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/625,386

(22) Filed: Jan. 22, 2007

Related U.S. Application Data
(62) Division of application No. 10/401,053, filed on Mar. 27, 2003, now abandoned.
(60) Provisional application No. 60/371,050, filed on Apr. 9, 2002.

(30) Foreign Application Priority Data
Mar. 26, 2002 (DE) 102 13 762.5

(51) Int. Cl. A61K 39/395; C07K 16/00

(52) U.S. Cl. 424/139.1; 530/387.9

(57) ABSTRACT

The invention relates to a novel costimulatory pathway mediated by a member of the semaphorin protein family, Sema4A, which is selectively expressed on the surface of dendritic cells. In addition, the invention relates to the use of Sema4A protein and protein derivatives in a method for the identification of immunomodulatory substances and to therapeutic applications making use thereof.

### APPLICATION DATA

This application is a divisional of U.S. application Ser. No. 10/401,053 filed Mar. 27, 2003 which claims priority to U.S. Provisional Application Ser. No. 60/371,050 filed on Apr. 9, 2002, the disclosure of which is incorporated by reference in its entirety.

### BACKGROUND OF THE INVENTION

The invention relates to a novel costimulatory protein selectively expressed on the surface of dendritic cells, to its use in a method for the identification of immunomodulatory substances, to functional derivatives thereof, to agents interfering with the respective costimulatory pathway, and to uses of said derivatives and interfering agents.

The generation of a T lymphocyte response is a complex process involving cell-cell interactions and production of soluble mediators (cytokines or lymphokines). Optimal activation of T lymphocytes is believed to require two cell-cell interaction signals: an antigen specific or clonal T cell receptor (TCR) signaling upon binding to the peptide-MHC on the antigen presenting cells (APCs), as well as a second, antigen non-specific “costimulatory” signal.

If a T cell encounters an antigen alone, without costimulation by costimulatory molecules, no significant amplification of an immune response against a given antigen occurs. Moreover, without costimulation, TCR engagement not only results in a failure to induce an immune response but leads to functional T-cell inactivation by either T cell anergy or apoptosis, resulting in tolerance. If the costimulatory signal is provided, the T cell will respond with clonal expansion specific for the stimulating antigen.

The quality and potency of an immune response depends on the type of APCs that process and present the antigen to T cells, the density of the peptide antigen/MHC ligand available for engagement of the TCR, and the provision of soluble and/or membrane-bound costimulatory signals by APCs at the time of T cell engagement and activation. APCs that provide the signals required for activation of T cells include monocytes/macrophages, B lymphocytes, and dendritic cells (DCs). Among these different types of APCs, DCs are considered as the most potent initiators of antigen-specific T cell responses in vivo. They most efficiently capture antigens and present them to T cells as MHC-peptide complexes in combination with various costimulatory signals. DCs have a distinct phenotype from activated macrophages and are classified into different subtypes capable of initiating distinct immune responses. In vitro they show an approximately 100-fold greater potency than macrophages to activate naive T cells.

A typical and the best characterized example for costimulatory molecules expressed on APCs such as DCs are the members of the so-called B7 family. This family includes B7, also known as B7-1 or CD80 and B7-2, also called CD86. They are members of the immunoglobulin (Ig) superfamily and comprise two extracellular Ig domains, an N-terminal variable (V)-like domain followed by a constant (C)-like domain. The ligands or counter-receptors of B7, expressed on the surface of T cells, are CD28 and CTLA-4. CD28 is a homodimeric glycoprotein of the Ig superfamily found on most mature human T cells that functions in T cell activation, is constitutively expressed on resting T cells and increases after activation. After signaling through the T cell receptor, ligation of CD28 induces T cells to proliferate and secrete IL-2. CTLA4 is a T cell surface molecule highly homologous to CD28 but is not expressed on resting T cells and appears following T cell activation.

However, the B7-CD28/CTLA-4 pathways do not account for all costimulatory activities. Indeed, interactions between tumor necrosis factor (TNF) and TNF receptor family members, including CD40-CD154, CD30-CD30L, CD27-CD70, 4-1 BB-4-1 BBL, RANK-RANKL (OPGL), and Ox40-Ox40L have also been demonstrated to be involved in T-cell costimulation through T cell-DC interactions. However, clearly, the framework of costimulatory molecules which determine the qualitative and quantitative T-cell responses have not yet been fully elucidated. Thus, T cell activation remains a highly complex field and, therefore, T cell function abnormalities can until now only be addressed very insufficiently by any therapeutic interventions.

A central function of the immune system is to distinguish foreign antigens, such as infectious agents, from self components of body tissues. The immune system normally acquires self tolerance (unresponsiveness to self) by clonal deletion of autoreactive T cells in the thymus in the perinatal period and by functional suppression of autoreactive T and B cells at later stages of development. Nevertheless, sometimes there is a failure in the maintenance of self tolerance, a failure to discriminate between self and non-self antigens, and an autoimmune response, characterized by the activation and clonal expansion of autoreactive lymphocytes and the production of autoantibodies, is produced against autologous antigens of normal body tissues. Although many autoimmune diseases are associated with autoantibodies and thus with B cells, T-cells may play an important role also in these pathological conditions as they can act on B-cell development and function.

Autoimmune diseases are multifactorial in origin and can be classified into organ-specific and nonorgan-specific (or systemic) autoimmune diseases. Clinical examples include: autoimmune hemolytic anemia, Hashimoto's thyroiditis, myasthenia gravis, Grave's disease, Goodpasture's syndrome, Crohn's disease, Guillain-Barre syndrome, psoriasis, myasthenia gravis, glomerulonephritis, autoimmune hepatitis, uveitis, type I (insulin-dependent) diabetes, rheumatoid arthritis, rheumatic fever, systemic lupus erythematosus (SLE), and multiple sclerosis.

Another group of diseases linked to enhanced T cell activity are allergies and asthma. Whereas T helper cells type 1 (Th1 cells) are assumed to play an important role in autoimmunity, allergy and asthma appear to be primarily associated with T helper cell type 2 (Th2 cell) responses.

On the other hand, a large number of pathological conditions are associated with insufficient immune responses. Immunodeficiencies may be primary or secondary. Primary immunodeficiency is classified into four main groups depending on which component of the immune system is deficient: B cells, T cells, phagocytic cells, or complement. Over 70 primary immunodeficiencies have been described. T cell deficiencies are e.g. DiGeorge anomaly, chronic mucocutaneous candidiasis, Nezelof syndrome, natural killer cell deficiency, and idiopathic CD4 lymphocytopenia. T cell as well as B cell reactions are hampered in severe combined immunodeficiency (SCID) which is probably the most important form of primary immunodeficiencies in terms of patient numbers.

The considerably more common secondary immunodeficiency is an impairment of the immune system resulting from an illness in a previously normal person. Of course, the most important diseases of this class are AIDS and AIDS related diseases. Other infectious diseases that may implicate immunodeficiencies are cytomegalovirus infection, infectious mononucleosis, acute bacterial disease, and severe mycobacterial or fungal disease.

Other causes for immunodeficiency are treatment with immunosuppressive agents such as radiation and immunosuppressive drugs. Of course, a medicament useful for the treatment of immunosuppressed conditions might also serve to stimulate the T cell response of a healthy individual.

Taking into account the above explanations on the molecular mechanisms of T cell activation, it might be expected that the antigen non-specific interaction between T cells and APCs is an important target for novel therapeutical, especially pharmacological approaches in the treatment of autoimmune diseases, primary and secondary immunodeficiencies, allergies and asthma.

The object of the present invention is therefore to provide novel means and methods for the modulation of T cell activation.

Especially, it is an object of the invention to provide substances, such as small molecules or biopharmaceuticals, for the preparation of medicaments for the regulation of T cell-APC interaction and especially T cell-DC interaction for the treatment of pathological conditions such as autoimmune diseases, immunodeficiency diseases, allergies and asthma.

It is a further object of the invention to provide a method for the identification of substances that modulate T cell-DC interaction and thereby T cell activation.

### SUMMARY OF THE INVENTION

The above object is solved by a first embodiment of the invention which provides an agent which modulates T cell-APC interaction, said agent being an isolated antibody or antibody derivative that selectively recognizes and binds to mammalian Sema4A protein. Preferably, the antibody or antibody derivative binds to human Sema4A protein.

As a second embodiment of the invention, there is provided an agent which modulates T cell-APC interaction, said agent being an isolated antibody or antibody derivative that selectively recognizes and binds to mammalian Tim-2 protein. Preferably, the antibody or antibody derivative binds to human Tim-2 protein.

The two afore-mentioned embodiments have in common that said antibodies or antibody derivatives inhibit the costimulatory effect of Sema4A protein on T cell activation.

Furthermore, there is provided according to the invention a pharmaceutical composition comprising an antibody or antibody derivative binding to Sema4A protein or Tim-2 as described above and an excipient, an adjuvant, a diluent, and/or a carrier.

Also encompassed by the present invention are: (i) the use of an antibody or antibody derivative binding to Sema4A protein or Tim-2 as described above for the preparation of a medicament for the treatment of a disease selected from autoimmune diseases, allergies and asthma, and (ii) methods for the treatment of a disease selected from autoimmune diseases, allergies and asthma comprising administering to a patient in need thereof a therapeutically effective amount of an antibody or antibody derivative as described above.

According to another aspect of the invention, there is provided an isolated and purified protein the sequence of which consists of SEQ ID NO:1. Furthermore, there are provided proteins having an amino acid sequence at least 95% identical, preferably at least 98% identical to SEQ ID NO:1, wherein said proteins have the biological function of acting as costimulatory molecules and wherein this biological function can be tested using the assays described in detail below.

SEQ ID NO:1 (human Sema4A protein according to the
invention):
MALPALGLDP WSLLGLFLFQ LLQLLLPTTT AGGGGQGPMP
RVKYYAGDER RALSFFHQKG LQDFDTLLLS GDGNTLYVGA
REAILALDIQ DPGVPRLKNM IPWPASDRKK SECAFKKKSN
ETQCFNFIRV LVSYNVTHLY TCGTFAFSPA CTFIELQDSY
LLPISEDKVM EGKGQSPFDP AHKHTAVLVD GMLYSGTMNN
FLGSEPILMR TLGSQPVLKT DNFLRWLHHD ASFVAAIPST
QVVYFFFEET ASEFDFFERL HTSRVARVCK NDVGGEKLLQ
KKWTTFLKAQ LLCTQPGQLP FNVIRQAVLL AADSPTAPHI
YGSSTSSGKV DGTRSSAVCA FSLLDIERAL KGEYKELNKE
TSRWTTYRGP ETNPRPGSCS VGPSSDKALT FMKDHFLMDE
QVVGTPLLVK SGVEYTRLAV ETAQGLDGHS HLVMYLGEEI
QLFPDPEPVR NLQLAPTQGA VFVGFSGGVW RVPRANCSVY
ESCVDCVLAR DPHCAWDPES RTCCLLSAPN LNSWKQDMER
GNPEWACASG PMSRSLRPQS RPQIIKEVLT VPNSILELPC
PHLSALASYY WSHGPAAVPE ASSTVYNGSL LLIVQDGVGG
LYQCWATENG FSYPVISYWV DSQDQTLALD PELAGIPREH
VKVPLTRVSG GAALAAQQSY WPHFVTVTVL FALVLSGALI
ILVASPLRAL RAAGSRFRAV RPCGPGEKAP LSREQHLQSP
KECRTSASDV DADNNCLGTE VA

A human protein named TANGO 265 and having an amino acid sequence (SEQ ID NO:2) similar to the above has previously been described (DERWENT® geneseq database, accession no. AAB66043). The degree of identity between SEQ ID NO:1 and SEQ ID NO:2 is 94% (Scoring matrix: BLOSUM62). No clear indication of the biological function of TANGO 265 protein has been given in the prior art. It may represent an allelic form or a splice variant of the now discovered human Sema4A protein.

SEQ ID NO:2 (human TANGO 265 protein):
MALPALGLDP WSLLGLFLFQ LLQLLLPTTT AGGGGQGPMP
RVRYYAGDER RALSFFHQKG LQDFDTLLLS GDGNTLYVGA
REAILALDIQ DPGVPRLKNM IPWPASDRKK SECAFKKKSN
ETQCFNFIRV LVSYNVTHLY TCGTFAFSPA CTFIELQDSY
LLPISEDKVM EGKGQSPFDP AHKHTAVLVD GMLYSGTMNN
FLGSEPILMR TLGSQPVLKT DNFLRWLHHD ASFVAAIPST
QVVYFFFEET ASEFDFFERL HTSRVARVCK NDVGGEKLLQ
KKWTTFLKAQ LLCTQPGQLP FNVIRHAVLL PADSPTAPHI
YAVFTSQWQV GGTRSSAVCA FSLLDIERVF KGKYKELNKE
TSRWTTYRGP ETNPRPGSCS VGPSSDKALT FMKDHFLMDE
QVVGTPLLVK SGVEYTRLAV ETAQGLDGHS HLVMYLGTTT
GSLHKAVVSG DSSAHLVEEI QLFPDPEPVR NLQLAPTQGA
VFVGFSGGVW RVPRANCSVY ESCVDCVLAR DPHCAWDPES
RTCCLLSAPN LNSWKQDMER GNPEWACASG PMSRSLRPQS
RPQIIKEVLA VPNSILELPC PHLSALASYY WSHGPAAVPE
ASSTVYNGSL LLIVQDGVGG LYQCWATENG FSYPVISYWV
DSQDQTLALD PELAGIPREH VKVPLTRVSG GAALAAQQSY
WPHFVTVTVL FALVLSGALI ILVASPLRAL RARGKVQGCE
TLRPGEKAPL SREQHLQSPK ECRTSASDVD ADNNCLGTEV
A

As a preferred embodiment, there is provided an isolated soluble mammalian Sema4A protein derivative comprising at least the extracellular sema domain of Sema4A protein and lacking at least a portion of the transmembrane domain of Sema4A protein.

In another preferred embodiment, there is provided a Sema 4A fusion protein comprising all or parts of a mammalian Sema4A protein fused to another protein or protein domain. Especially preferred is a fusion protein having the sequence SEQ ID NO:3 or a fusion protein essentially as shown in SEQ ID NO:3 but comprising the human counterparts of the respective protein components.

SEQ ID NO:3 (mSema4A-Fc):
MALPSLGQDS WSLLRVFFFQ LFLLPSLPPA SGTGGQGPMP
RVKYHAGDGH RALSFFQQKG LRDFDTLLLS DDGNTLYVGA
RETVLALNIQ NPGIPRLKNM IPWPASERKK TECAFKKKSN
ETQCFNFIRV LVSYNATHLY ACGTFAFSPA CTFIELQDSL
LLPILIDKVM DGKGQSPLTL FTSTQAVLVD GMLYSGTMNN
FLGSEPILMR TLGSHPVLKT DIFLRWLHAD ASFVAAIPST
QVVYFFFEET ASEFDFFEEL YISRVAQVCK NDVGGEKLLQ
KKWTTFLKAQ LLCAQPGQLP FNIIRHAVLL PADSPSVSRI
YAVFTSQWQV GGTRSSAVCA FSLTDIERVF KGKYKELNKE
TSRWTTYRGS EVSPRPGSCS MGPSSDKALT FMKDHFLMDE
HVVGTPLLVK SGVEYTRLAV ESARGLDGSS HVVMYLGTST
GPLHKAVVPQ DSSAYLVEEI QLSPDSEPVR NLQLAPAQGA
VFAGFSGGIW RVPRANCSVY ESCVDCVLAR DPHCAWDPES
RLCSLLSGST KPWKQDMERG NPEWVCTRGP MARSPRRQSP
PQLIKEVLTV PNSILELRCP HLSALASYHW SHGRAKISEA
SATVYNGSLL LLPQDGVGGL YQCVATENGY SYPVVSYWVD
SQDQPLALDP ELAGVPRERV QVPLTRVGGG ASMAA - Fc

Also according to this aspect of the invention there is provided a pharmaceutical composition that, when administered to a subject, stimulates T cell mediated immune responses in said subject, wherein said pharmaceutical composition comprises: (i) a pharmaceutically active component selected from the group consisting of: the Sema4A protein according to SEQ ID NO:1, a functional fragment or derivative thereof, such as a soluble mammalian Sema4A protein derivative as outlined above or a mammalian Sema4A fusion protein as described above, and (ii) one or more components selected from the group consisting of: excipients, adjuvants, diluents and carriers.

In the context of this embodiment, the pharmaceutical composition shows the physiological effect of stimulating T cell mediated immune responses. This effect can be assessed e.g. by an assay comprising the steps: (i) stimulating naive CD4+ T cells with immobilized anti-CD3 antibodies and anti-CD28 antibodies in the presence or absence of said pharmaceutically active component and (ii) measuring the activation of the thus treated T cells by measuring T cell proliferation or IL-2 secretion. If in the presence of the compound T cell proliferation or IL-2 secretion is increased as compared to assays in which said compound is not included, this compound is classified in the context of this invention as a compound having the biological effect of stimulating T cell mediated immune responses.

Also within the scope of this invention are: (i) the use of a pharmaceutically active substance selected from the group consisting of: the Sema4A protein according to SEQ ID NO:1, a functional fragment or derivative thereof, especially a soluble mammalian Sema4A protein derivative as outlined above and a mammalian Sema4A fusion protein as described above, wherein said pharmaceutically active substance has the biological effect of stimulating T cell mediated immune responses, for the preparation of a medicament for the treatment of primary or secondary immunodeficiencies or for the stimulation of normal T cell responses, as well as (ii) a method for the treatment of a disease selected from primary or secondary immunodeficiencies or for the stimulation of T cell responses comprising administering to a patient in need thereof a therapeutically effective amount of a substance selected from the afore-mentioned group.

A further embodiment of the invention is represented by a mammalian Sema4A protein derivative reactive with Tim-2 antigen present on the surface of T cells.

According to a further aspect of the invention there is provided a method of identifying a compound capable of modulating T cell mediated immune responses in a mammal including man comprising the steps: (i) preparing a candidate compound, (ii) contacting a T cell expressing a Sema4A receptor on its surface with said candidate compound, (iii) contacting said T cell with a Sema4A agent under conditions suitable to activate said T cell and (iv) determining if said candidate compound modulates the activation of said T cell, wherein said Sema4A agent is selected from the group consisting of: a mammalian Sema4A protein, the human Sema4A protein as given by SEQ ID NO:1, a functional fragment or derivative thereof, a soluble mammalian Sema4A protein derivative as outlined above, a mammalian Sema4A fusion protein as described above, and a cell expressing Sema4A protein or a Sema4A protein derivative on its surface, wherein said Sema 4A protein derivative comprises at least the extracellular sema domain of Sema4A protein.

In this method, the modulation of the activation of said T cell is preferably determined by measuring T cell proliferation or the secretion of a cytokine, e.g. interleukin-2, interferon-gamma, and interleukin-4, by T cells into the culture medium.

In a preferred embodiment of the invention, bone marrow-derived dendritic cells are used as the Sema4A agent. Also preferably, T cells are contacted with an anti-CD3 antibody and optionally an anti-CD28 antibody in order to create conditions suitable to activate T cells. Furthermore, the T cells expressing a Sema4A receptor on their surface are preferably CD4+ T cells prepared from splenocytes.

In another preferred embodiment, a Sema4A fusion protein and more preferably the Sema4A-Fc fusion protein of SEQ ID NO:3 is used in the above method. Especially preferred is the use of a functional human Sema4A protein or protein derivative.

In the methods mentioned above, the active substances modulate the T cell mediated immune response by interacting with the DC-T cell costimulatory pathway.

According to a further aspect of the invention, substances selected from the group consisting of: anti-Sema4A and anti-Tim-2 antibodies or antibody derivatives, mammalian Sema4A proteins, the human Sema4A protein as given by SEQ ID NO:1, a functional fragment or derivative thereof, a soluble mammalian Sema4A protein derivative as outlined above, and compound identified by a method as described in the afore-mentioned embodiments, are used for the investigation of T cell costimulatory pathways or for the preparation of a medicament for the treatment of diseases linked to T cell activation abnormalities. T cell responses can be modulated in vitro and in vivo by administering to the T cells a substance selected from the above group.

### BRIEF DESCRIPTION OF THE FIGURES

FIG. 1 shows the sequence alignment of human (upper line; SEQ ID NO:1) and mouse (bottom line; SEQ ID NO:4) Sema4A. The middle line indicates identical amino acid residues. Predicted signal sequence (small-dashed line), Sema domain (solid line), Ig-like domain (large-dashed line), and transmembrane region (bold line) are indicated.

FIG. 2 shows the results of the experiment of Example 5. Treatment with anti-Sema4A antibodies blocked the development of experimental autoimmune encephalomyelitis (EAE). A: EAE clinical disease course in mice treated with anti-Sema4A (open circles) or control rat IgGs (closed circles). B: In vitro responses (proliferation, IL-4 and IFN-γ production) of CD4+ T cells stimulated with myelin oligodendrocyte glycoprotein (MOG)-peptide.

### DETAILED DESCRIPTION OF THE INVENTION

Before the invention is described in greater detail, it should be noted that in the specification and the appended claims, the singular forms “a”, “an” and “the” include plural reference unless the context clearly dictates otherwise. Thus, for example, reference to “a cell line” is a reference to one or more cell lines, and the like. The amino acid abbreviations are according to the standard one or three letter code. Other abbreviations used in the specification and the claims are explained at the site of their first appearance.

Upon studying the molecular mechanisms of lymphocyte activation, the inventors surprisingly identified a novel type of T cell costimulatory pathway mediated by a member of the semaphorin family. The semaphorin family includes a large number of phylogenetically conserved proteins and comprises secreted and transmembrane proteins carrying a large “sema domain” (approximately 500 amino acid residues) in their extracellular regions. Many semaphorins of the secreted-type have been shown to be involved in axon guidance acting as chemorepulsion factors or delivering guidance cues to migrating axons during neuronal development. Surprisingly, it can now be established that semaphorins are also crucially involved in T-cell costimulation including pathogenic immune reactions.

Especially, the inventors identified the semaphorin family member Sema4A as an important costimulatory molecule. Sema 4A is preferentially expressed on the cell surface of DCs and has a potent costimulatory activity including in vitro T cell activation and in vivo generation of antigen-specific T cells. Furthermore, it can be demonstrated that Sema4A is an important target for the treatment of autoimmune diseases. Administration of anti-Sema4A antibodies effectively inhibits the development of experimental autoimmune encephalomyelitis (EAE).

Also in the context of this invention, the inventors were able to identify the counter-receptor for Sema4A on T cells. First, it became clear that neuropilins and plexins, which are the receptors for semaphorins involved in axon guidance in the nervous system, are not involved in Sema4A mediated costimulatory pathways. Then, surprisingly, the inventors identified an already known T cell surface antigen, Tim-2, as a receptor for Sema4A on T cells. Tim-2 was known to be expressed on activated T cells but its function was unknown. Tim-2 belongs to the Tim gene family characterized by their expression on the cell surface of T cells and the conserved immunoglobulin and mucin domains. Recently, Tim-1 has been implicated in Th2 cell responses as an airway hyperreactivity regulatory gene. Furthermore, Tim-3 has been demonstrated to be expressed exclusively on Th1 cells and to be crucially involved in Th1 cell responses and macrophage activation.

Based on these and further results outlined in the Examples, the following paradigm is suggested. Shortly after T cell activation, bindings sites for Sema4A (putative receptor: Tim-2) are induced on the surface of the activated T cells. Subsequently, the binding sites are engaged by Sema4A expressed on DCs, leading to influence T cell activation in T cell-DC contacts, which might strengthen the activation of TCR signals, resulting in promoting differentiation of T cells into functional effector cells. These findings suggest the importance of Sema4A in early phases of T cell activation. Indeed, in vivo T cell priming was enhanced by administration of Sema4A-Fc. In addition, anti-Sema4A significantly inhibited the generation of antigen-specific T cells. These results also support the notion that Sema4A is involved in the initial priming stage between T cells and DCs.

Based on the above findings, there is provided according to one aspect of the invention a method for the identification of modulators using Sema4A, Tim-2 or the interaction between Sema4A and Tim-2 as target.

To test compounds for a modulating effect on Sema4A-T cell interaction, naive CD4+ T cells can be incubated with respective test compounds and stimulated with immobilized anti-CD3 antibodies, anti-CD28 antibodies and e.g. Sema4A-Fc. As a control, the same assays are performed without a test compound being added. As a readout, T cell proliferation and interleukin-2 (IL-2) production can be determined. Alternatively, interferon-gamma (IFN-γ) or IL-4 secretion can be measured. The skilled person will set the remaining parameters of the assay system in an appropriate manner. One example for a suitable assay system for the assessment of Sema4A-T cell interaction and the modulation thereof by test compounds is described in detail in Examples 6 and 7 and the Methods below.

The above assay can be performed as a high throughput screening (HTS) method. HTS relates to an experimental setup wherein a large number of compounds are tested simultaneously. Preferably, said HTS setup may be carried out in microplates, may be partially or fully automated and may be linked to electronic devices such as computers for data storage, analysis, and interpretation using bioinformatics. Preferably, said automation may involve robots capable of handling large numbers of microplates and capable of carrying out several thousand tests per day. Preferably, a test compound which is known to show the desired modulating or inhibitory function will also be included in the assay as a positive control. The term HTS also comprises ultra high throughput screening formats (UHTS). Preferably, said UHTS formats may be carried out using 384- or 1536-well microplates, sub-microliter or sub-nanoliter pipettors, improved plate readers and procedures to deal with evaporation. HTS methods are described e.g. in U.S. Pat. No. 5,876,946 and U.S. Pat. No. 5,902,732. The expert in the field can adapt the method described above to an HTS or UHTS format without the need for carrying out an inventive step.

Compounds identified by the above screening assay are useful for the further investigation of T cell costimulatory pathways, for the preparation of medicaments for the treatment of diseases linked to T cell activation abnormalities and ultimately for the treatment of such diseases.

The diseases that can be addressed by compounds modulating the Sema4A costimulatory pathway include autoimmune diseases such as autoimmune hemolytic anemia, Hashimoto's thyroiditis, myasthenia gravis, Grave's disease, Goodpasture's syndrome, Crohn's disease, Guillain-Barre syndrome, psoriasis, myasthenia gravis, glomerulonephritis, autoimmune hepatitis, uveitis, type I (insulin-dependent) diabetes, rheumatoid arthritis, rheumatic fever, systemic lupus erythematosus (SLE), and multiple sclerosis. Further examples are allergic diseases and asthma. In these cases, a compound inhibiting the Sema4A costimulatory pathway and, thereby, preventing T cell activation, may serve to inhibit hyperreactivity of the T (helper) cell system.

On the other hand, primary and secondary immunodeficiencies can be addressed by an activation of the T (helper) cell system. Primary T cell immunodeficiencies are e.g. DiGeorge anomaly, chronic mucocutaneous candidiasis, Nezelof syndrome, natural killer cell deficiency, idiopathic CD4 lymphocytopenia and SCID. Secondary immunodeficiencies are AIDS and AIDS related diseases, cytomegalovirus infection, infectious mononucleosis, acute bacterial disease, and severe mycobacterial or fungal disease. Other causes for immunodeficiency are treatment with immunosuppressive agents such as radiation and immunosuppressive drugs. Finally, it is expected that in certain cases a general stimulation of the immune response of a healthy organism can be useful. The stimulation of the immune response might be achieved by an antigen nonspecific T cell stimulation. In all these cases, a compound activating and/or enhancing the costimulatory pathway will be useful.

Indeed, based on the above rationale, the inventors successfully treated an autoimmune disease by administering an agent that inhibits the Sema4A costimulatory pathway, in this case an anti-Sema4A antibody. Thus, according to another important aspect of the present invention, there are provided agents useful for the treatment of autoimmune diseases including but not limited to: autoimmune hemolytic anemia, Hashimoto's thyroiditis, myasthenia gravis, Grave's disease, Goodpasture's syndrome, Crohn's disease, Guillain-Barre syndrome, psoriasis, myasthenia gravis, glomerulonephritis, autoimmune hepatitis, uveitis, type I (insulin-dependent) diabetes, rheumatoid arthritis, rheumatic fever, systemic lupus erythematosus (SLE), and multiple sclerosis.

Although blocking of the Sema4A costimulatory pathway was achieved by administering monoclonal anti-Sema4A antibody SK31 (described in more detail in Example 5 and the Methods), it will be acknowledged that similar results can be achieved upon administering other inhibitory agents such as e.g. small molecules as identified in the screening assays above or biopharmaceuticals such as e.g. other monoclonal antibodies, polyclonal antibodies and antibody derivatives.

Thus, based on the above-mentioned results obtained with antibody SK31, an important embodiment of the invention are anti-Sema4A antibodies and antibody derivatives which recognize and bind to Sema4A, thereby blocking the Sema4A mediated costimulatory pathway. The antibodies are preferably directed against the human Sema4A protein, may be obtained from any species and may be polyclonal or monoclonal antibodies. Especially preferred are humanized monoclonal antibody proteins. The binding of such antibodies or antibody derivatives and the inhibiting effect thereof on T cell activation can be assessed as described below in the Examples and Methods.

The term “antibody or antibody derivative” is meant to include e.g. Fab fragments (Fab=Fragment antigen-binding) which include the variable regions of both chains held together by the respective adjacent constant regions. Fab fragments may be formed by protease digestion, e.g. with papain, from conventional antibodies, but similar Fab fragments may also be produced in the mean time by genetic engineering. Also included in this term are F(ab′)₂ fragments, which may be prepared by proteolytic cleavage with pepsin.

Using genetic engineering methods it is possible to produce shortened antibody fragments which consist only of the variable regions of the heavy (VH) and of the light chain (VL). These are referred to as Fv fragments (Fragment variable=fragment of the variable part). Since these Fv-fragments lack the covalent bonding of the two chains by the cysteines of the constant chains, the Fv fragments are often stabilised. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins of this kind known from the prior art are described in Huston et al. (1988, PNAS 16:5879-5883). In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerisation of the scFv, scFv were prepared as fusion proteins with multimerisation domains. The multimerisation domains may be, e.g. the CH3 region of an IgG or a coiled coil structure (helix structures) such as leucine-zipper domains.

However, there are also strategies in which the interaction between the VH/VL regions of the scFv itself is used for the multimerisation (e.g. di-, tri- and pentabodies). Thus, also meant to be encompassed by the above term are so-called diabodies. Diabody means a bivalent homodimeric scFv derivative (Hu et al., 1996, PNAS 16: 5879-5883). The shortening of the Linker in an scFv molecule to 5-10 amino acids leads to the formation of homodimers in which an interchain VH/VL-superimposition takes place. Diabodies may additionally be stabilised by the incorporation of disulphide bridges. Examples of diabody-antibody proteins from the prior art can be found in Perisic et al. (1994, Structure 2:1217-1226).

Another sort of antibody derivative is represented by so-called minibodies. These are bivalent, homodimeric scFv derivatives consisting of a fusion protein which contains the CH3 region of an immunoglobulin, preferably IgG, most preferably IgG1 as the dimerisation region which is connected to the scFv via a hinge region (e.g. also from IgG1) and a linker region. The disulphide bridges in the hinge region are mostly formed in higher cells and not in prokaryotes. Examples of minibody-antibody proteins from the prior art can be found in Hu et al. (1996, Cancer Res. 56:3055-61). By triabody the skilled person means a trivalent homotrimeric scFv derivative (Kortt et al. 1997 Protein Engineering 10:423-433). ScFv derivatives wherein VH-VL are fused directly without a linker sequence lead to the formation of trimers. The skilled person will also be familiar with miniantibodies having a bi-, tri- or tetravalent structure wherein the multimerisation is carried out by di-, tri- or tetrameric coiled coil structures (Pack et al., 1993 Biotechnology 11: 1271-1277; Lovejoy et al. 1993 Science 259:1288-1293; Pack et al., 1995 J. Mol. Biol. 246:28-34).

According to another aspect, the invention relates to pharmaceutical compositions comprising said antibody or antibody derivative as active substance and to the use of said antibody or antibody derivative for the preparation of a medicament for the treatment of diseases such as autoimmune diseases, allergic diseases and asthma as already outlined above in detail. A further aspect of the invention is the use of said antibody or antibody derivative in a method for the treatment of said autoimmune diseases, allergic diseases and asthma. Suitable excipients, adjuvants, diluents and carriers that may be used in the pharmaceutical compositions are known in the art. Examples can be taken e.g. from the handbook: Gennaro, Alfonso R.: “Remington's Pharmaceutical Sciences”, Mack Publishing Company, Easton, Pa., 1990.

According to a further aspect of the invention and based on the above-mentioned finding that Tim-2 is a counter-receptor for Sema4A, the costimulatory pathway mediated by Sema4A can also be blocked by administering anti-Tim-2-antibodies, respective antibody derivatives as outlined above and small molecules blocking Sema4A-Tim-2-interaction.

According to another aspect of the invention, there is provided a method for regulating immune responses by contacting T cells with Sema4A protein, such as human Sema4A or functional fragments or derivatives thereof. The term “functional fragment or derivative” means a protein having part or all of the primary structure of a mammalian, preferably human Sema4A and possessing at least the biological property of binding to the Sema4A receptor on T cells.

Preferably, said functional fragment or derivative is a soluble Sema4A protein, more preferably a soluble human Sema4A protein. A recombinant soluble Sema4A protein can be produced by standard cloning techniques known in the art, e.g. by deleting all or parts of the transmembrane domain of natural Sema4A protein (functional fragment).

A preferred example of a functional derivative is a fusion protein construct including at least a portion of the extracellular domain of Sema4A protein and another protein, e.g. human immunoglobulin C gamma 1, that alters the solubility, binding affinity and/or valency of Sema4A protein.

An example of a soluble functional derivative is Sema4A-Fc having the amino acid sequence according to SEQ ID NO:3.

Also according to this aspect of the invention there is provided a pharmaceutical composition that, when administered to a subject, stimulates T cell mediated immune responses in said subject, said pharmaceutical composition comprising: (i) a pharmaceutically active component selected from the group consisting of: the Sema4A protein according to SEQ ID NO:1, a functional fragment and a functional derivative of mammalian Sema4A protein as outlined above, and (ii) one or more components selected from the group consisting of: excipients, adjuvants, diluents and carriers.

In the context of this embodiment, the pharmaceutical composition shows the physiological effect of stimulating T cell mediated immune responses. This effect can be assessed e.g. by an assay comprising the steps: (i) stimulating naive CD4+ T cells with immobilized anti-CD3 antibodies and anti-CD28 antibodies in the presence or absence of said pharmaceutically active component and (ii) measuring the activation of the thus treated T cells by measuring T cell proliferation or IL-2 secretion. If in the presence of the compound, T cell proliferation or IL-2 secretion is increased as compared to assays in which said compound is not included, this compound is classified in the context of this invention as a compound having the biological effect of stimulating T cell mediated immune responses. As shown in the Examples, a functional Sema4A derivative as defined above causes a strong in vitro T cell activation when administered together with anti-CD3 and/or anti-CD28 antibodies. A cytokine may be added in order to optimize stimulation of T cells.

Also within the scope of this invention are: (i) the use of a pharmaceutically active substance selected from the group consisting of: the Sema4A protein according to SEQ ID NO:1, a functional fragment and a functional derivative of mammalian Sema4A protein as outlined above, wherein said pharmaceutically active substance has the biological effect of stimulating T cell mediated immune responses, for the preparation of a medicament for the treatment of primary or secondary immunodeficiencies or for the stimulation of normal T cell responses, as well as (ii) a method for the treatment of a disease selected from primary or secondary immunodeficiencies or for the stimulation of T cell responses comprising administering to a patient in need thereof a therapeutically effective amount of a substance selected from the afore-mentioned group.

The functional mammalian Sema4A protein derivative encompassed by the present invention is expected to specifically bind to Tim-2 antigen present on the surface of T cells. Thus, the invention also provides a method for treating immune system diseases by administering Sema4A protein, functional fragments or derivatives, including soluble human Sema4A fusion proteins, to react with T cells by binding to the Tim-2 antigen.

In yet another embodiment, a method for inhibiting T cell proliferation in graft versus host disease is provided wherein Tim-2 positive T cells are reacted with Sema4A, preferably with a soluble human Sema4A protein fragment or derivative, to bind to the Tim-2 receptor, and an immunosuppressant is administered.

### EXAMPLES

### Example 1

### Isolation of Mouse and Human Sema4A

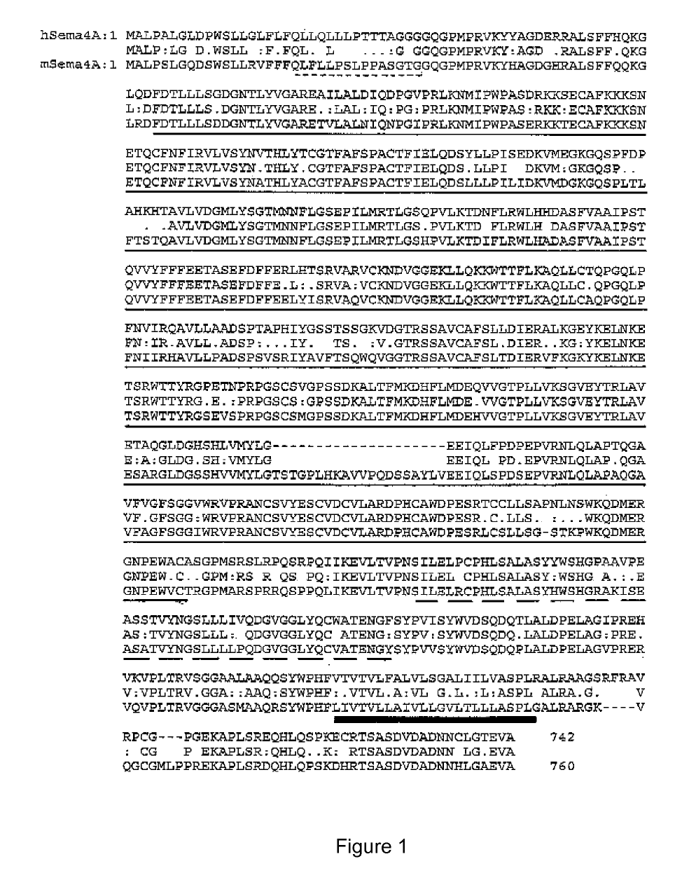

In an effort to understand semaphorins expressed in DCs, PCR cloning using degenerated oligonucleotide primers based on conserved motifs among members of the semaphorin family has been performed. Thereby, a cDNA fragment of Sema4A which has been classified into the class IV of the semaphorin family has been identified. Sema4A was originally identified as semB of which expression has been observed in mouse embryos, although no information on its function has been reported. Since the human homologue of Sema4A was not identified, the database of National Center for Biotechnology Information (NCBI) was searched with the nucleotide sequence of mouse Sema4A. Based on the resulting incomplete nucleotide sequence of human Sema4A, a full-length of complementary cDNA from a human brain cDNA library was isolated and its complete nucleotide sequence was determined. As shown in FIG. 1, a 78% identity in the amino acid sequence between the human and mouse Sema4A was found. The amino acid sequence of human Sema4A differs slightly from that of mouse Sema4A in that it has a shorter (20 amino acids) extracellular region. Based on its structural features, Sema4A has been classified into the class IV of the semaphorin family. The amino-terminal signal sequence is followed by a sema domain, an Ig-like domain, a hydrophobic transmembrane region, and a cytoplasmic tail. Interestingly, cysteine residues in the semaphorin domain are conserved between Sema4A and CD100, another member of the semaphorin family.

### Example 2

### Expression of Sema4A

Although the expression of mouse Sema4A during embryonic development has been reported, its expression profiles in the adult tissues have not been reported. To exclude the possible cross hybridization among the semaphorin family in the case of northern blot analysis, RT-PCR for analysis of Sema4A-expression using Clontech, BD Biosciences, Palo Alto, Calif.'s mouse multiple tissue cDNA panels was performed. The results were as follows: Sema4A was expressed in a broad range of tissues with prominent levels in the brain, spleen, lung, kidney and testis. In addition, the expression of Sema4A was not detectable by embryo day 7 but it became detectable and gradually increased during embryonic development, of which embryonic expression profiles are consistent with those reported previously.

To investigate the functions and expression of Sema4A in the immune system, recombinant soluble mouse Sema4A protein consisting of the putative extracellular region of mouse Sema4A fused with human IgG1 Fc (Sema4A-Fc) was prepared. Identity of the product obtained was shown by SDS-PAGE. Two micrograms of purified Sema4A-Fc protein was separated by gradient PAGE (4%-20%) in the presence of 0.1% SDS under reducing conditions or non-reducing conditions and visualized by silver staining. A band of approximately 120 kDa was observed for Sema4A-Fc under reducing conditions, and dimer formation was apparent under non-reducing conditions.

To analyze the cell surface expression of Sema4A on various types of cells, anti-mouse Sema4A monoclonal antibodies (anti-Sema4A) were produced by immunizing rats with Sema4A-Fc and screening hybridomas with mouse Sema4A-expressing CHO cell transfectants (Sema4A-CHO) by flow cytometric analysis. It could be confirmed that anti-Sema4A (SK31, rat IgG2a) specifically bound to Sema4A-CHO but not to either control CHO cell transfectants with neomycin resistance plasmid alone (CHOneo) or CD100-expressing CHO cells (CD100-CHO). As expected from the cloning methodology of mouse Sema4A cDNA, flow cytometric analysis using anti-Sema4A confirmed that Sema4A was expressed abundantly on the surface of bone marrow derived and splenic DCs. Its expression was moderately detected on the surface of B cells. However, its expression was not detected on the surface of T cells where CD100 is abundantly expressed.

### Example 3

### Involvement of Sema4A in T Cell Activation

To test whether Sema4A has an effect on T cell activation, CD4+ T cells were stimulated with immobilized anti-CD3 plus anti-CD28 in the presence or absence of Sema4A-Fc. As a result, Sema4A-Fc enhanced anti-CD3 induced T cell proliferation and IL-2 production.

Next it was examined whether Sema4A promotes the differentiation of T cells into Th1-like or Th2-like effector populations under the respective culture conditions. Naive T cells were cultured with anti-CD3 plus anti-CD28 in the presence of IL-12 plus anti-IL-4 (Th1 conditions) or IL-4 (Th2 conditions) for 6 days, and the resulting cells were re-stimulated with anti-CD3 plus anti-CD28 for 48 hr. The production of IFN-γ or IL-4 was measured by ELISA. In the presence of Sema4A-Fc, the induction of either IFN-γ or IL-4 producing cells was significantly enhanced compared to that in the absence of Sema4A-Fc. However, Sema4A-Fc did not have any effects on Th1-like or Th2-like effector populations. These findings suggest that Sema4A is important for the early phases of T cell activation.

In this context, it is noteworthy that, in the absence of anti-CD28, Sema4A was not effective on anti-CD3 induced T cell responses. In the presence of anti-CD28, Sema4A exhibited marked effects on anti-CD3 induced T cell activation. These findings suggest that Sema4A costimulates T cells in combination with other costimulatory molecules, in particular, B7 family members (CD80 and CD86), expressed on DCs.

Furthermore, it was examined whether Sema4A-Fc has an effect on mixed lymphocyte reactions (MLR) between allogeneic T cells and DCs. Bone marrow derived DCs on a C57BL/6 background were utilized as stimulators in MLR with CD4+ T cells isolated from the spleen on a BALB/c background as responders. Sema4A-Fc significantly enhanced T cell proliferation in the MLR. The production of IL-2 in the culture supernatants was also enhanced by Sema4A-Fc. Furthermore, even when DCs which were fully-maturated by the treatment of anti-CD40 and then fixed with paraformaldehyde were used as stimulators for MLR, Sema4A-Fc exhibited enhancing effects on the MLR, indicating that Sema4A directly acts on T cells. Collectively, these results indicate that Sema4A expressed on DCs plays a role in T cell activation through T cell-DC interactions.

A class IV semaphorin, CD100, has previously been shown to be involved in the activation of B cells and DCs. We thus tested whether Sema4A-Fc has an effect on B cells (proliferation) and DCs (maturation) as it is the case for CD100. Small resting B cells purified from C57BL/6 mice were stimulated with or without anti-CD40 and IL-4 in the presence of either Sema4A-Fc or CD100-Fc for 72 hr. Cells were pulsed with [³H]thymidine. Although CD100-Fc significantly enhanced CD40-induced proliferation of B cells and IL-12 production of DCs, Sema4A-Fc did not show such effects on these cells. These results indicate that Sema4A plays a distinct role from CD100.

### Example 4

### Immunostimulatory Effect of Sema4A

To determine whether Sema4A plays a role in antigen-specific T cell responses in vivo, mice were immunized with keyhole limpet haemocyanin (KLH) in complete Freund's adjuvant (CFA) subcutaneously in the hind foot pad and then treated with Sema4A-Fc every 4 days intravenously. Five days after immunization, CD4+ T cells were prepared from the draining lymph nodes, and were tested in vitro for antigen-specific responses of T cells. A dramatic increase in the proliferation and the production of both IL-4 and IFN-γ of CD4+ T cells from draining lymph nodes was observed in mice treated with Sema4A-Fc but not with control human IgG1. These findings indicate that Sema4A has an enhancing effect on the in vivo priming of antigen-specific T cells.

### Example 5

### Treatment of Autoimmune Diseases by Blocking the Sema4A Costimulatory Pathway

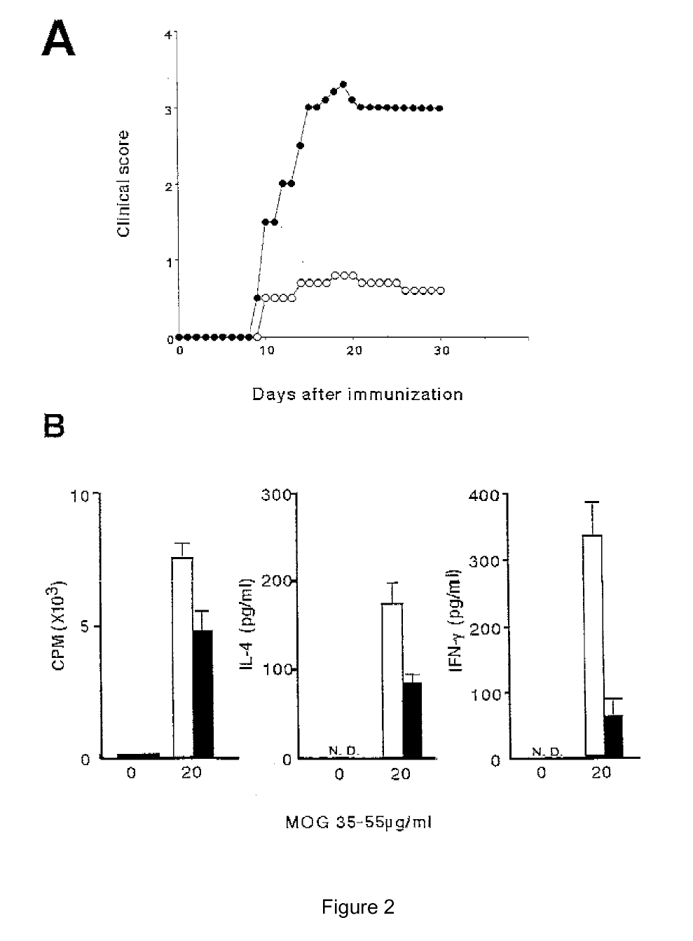

Subcutaneous immunization with a peptide derived from myelin oligodendrocyte glycoprotein (MOG) together with pertussis toxin uniformly and reproducibly induces experimental autoimmune encephalomyelitis (EAE) as described previously. Therefore, involvement of Sema4A in physiological and pathological immune responses was examined using this model. Mice were immunized wiTh 100 μg of MOG-peptide in CFA together with pertussis toxin on day 0 and scored for clinical signs of EAE as described previously. Mice were treated intraperitoneally wiTh 100 μg of antibodies (anti-Sema4A or control rat IgGs) every day from day 0 until day 4 post-immunization. The mean clinical score of each group was assessed and plotted against the time after immunization. As shown in FIG. 2A, the development of EAE was significantly suppressed in mice treated with anti-Sema4A compared to that in mice treated with control rat IgGs.

To determine the mechanisms responsible for the resistance to EAE in mice treated with anti-Sema4A, CD4+ T cells were prepared five days after immunization from the draining lymph nodes and stimulated for 72 hr with various concentrations of MOG-peptide in the presence of irradiated splenocytes of C57BL/6 mice. Proliferation was assessed during the final 12 hr of culture by pulsing wiTh2 μCi [³H] thymidine. IL-4 and IFN-γ production in the culture supernatants were measured by ELISA. As shown in FIG. 2B, antigen-specific T cell responses were severely impaired in mice treated with anti-Sema4A, indicating that the generation of antigen-specific T cells is inhibited by administration of anti-Sema4A. This result suggests that Sema4A is crucially involved in physiological and pathological cellular immune responses.

### Example 6

### Screening Assay for the Identification of Compounds Having a Modulating Effect on Sema4A-T cell Interaction

To test compounds for a modulatory effect on Sema4A-T cell interaction and thereby on T cell activation, naive CD4+ T cells are incubated with respective test compounds and are stimulated with immobilized anti-CD3 plus anti-CD28 antibodies and Sema4A-Fc. Controls are performed without addition of the test compound. As readout, T cell proliferation and IL-2 production are measured.

Compounds which result in increased T cell proliferation or IL-2 production are classified as T cell stimulation activators, whereas compounds resulting in a decreased T cell proliferation or IL-2 production are classified as T cell stimulation inhibitors. They are useful as lead compounds for the development of small molecule pharmaceuticals for the treatment of e.g. autoimmune diseases, allergies, or asthma (inhibitors) or primary and secondary immunodeficiencies (activators).

### Example 7

### HTS-Assay

The assay is performed essentially as described in Example 6 with the exception that it is carried out under HTS conditions described above and sufficiently known in the art.

### Example 8

### Identification of the Sema4A Counter-Receptor on T cells

To determine the expression of the putative counter-receptor of Sema4A (known hereafter as Sema4A receptor), various cells (splenic B cells, bone-marrow derived DCs, splenic T cells or EL-4 cells) were stained with biotinylated Sema4A-Fc. The binding of biotinylated Sema4A-Fc was not detected on primary T cells, B cells or DCs. Even after the B cells and the DCs were stimulated with anti-CD40, the binding of biotinylated Sema4A-Fc was not detected. However, binding of biotinylated Sema4A-Fc became detectable on T cells following Concanavalin A (ConA) stimulation, suggesting the expression of Sema4A receptor on activated T cells. Furthermore, Sema4A-binding sites were observed on the surface of some T cell lines, such as EL-4 cells. These results suggest the expression of Sema4A receptor on activated T cells.

For expression cloning of the Sema4A receptor, a cDNA library from EL-4 cells was constructed. Plasmid DNA from the library was introduced into COS7 cells. The transfected COS7 cells were allowed to bind biotinylated Sema4A-Fc or biotinylated human immunoglobulin Fc fractions followed by magnetic beads conjugated with streptavidin. Cells binding Sema4A-Fc were enriched by magnetic sorting. A discrete band corresponding to a 960 bp insert appeared after a third round of sorting, whereas no bands were apparent with cells binding human immunoglobulin Fc fractions. Upon sequencing of the 960 bp cDNA insert of these clones, the full-length of cDNAs encoding Tim-2 was identified.

Methods

Isolation of cDNA Fragments of Mouse Sema4A

Based on the sequences conserved among members of the Semaphorin family, degenerate 5′-AARTGGACIACITTYYTIAARGC-3′ (SEQ ID NO:5) and 5′-TCCCAIGCRCARTRIGGRTC-3′ (SEQ ID NO:6; R=G or A; Y=T or C; I=Inosine) oligonucleotides were used for PCR amplification, using cDNA prepared from bone marrow derived DCs of CD100-deficient mice (94° C. for 1 min; 55° C. for 1 min; 72° C. for 1 min; 30 cycles). Amplification products were cloned into a T/A vector (Novagen, Madison, Wis.) and sequenced. A BLAST search of a mouse EST database of the National Center for Biotechnology Information identified a cDNA of mouse Sema4A (X85991). Using this sequence, a full-length cDNA was cloned from a cDNA library generated from bone marrow derived DCs by PCR using primers containing a sense sequence including a SalI site 5′-AGGTCGACCCATCTGGTGACCATCTCAGGCTGACCATGGC-3′ (SEQ ID NO:7) and an antisense sequence including a NotI site and FLAG (DYKDDDDK; SEQ ID NO:8) sequence 5′-ATGCGGCCGCTTACTTGTCATCGTCGTCCTTGTAGTCAGCCACTTCGGCGCCCAGATGGT TG-3′ (SEQ ID NO:9). The resulting SalI-NotI fragments were cloned into pEFBos vector.

RT-PCR Analysis for Expression of Sema4A

The expression profiles of Sema4A in mouse tissues were analysed by RT-PCR using mouse multiple tissue cDNA panels (Clontech, BD Biosciences, Palo Alto, Calif.). Based on the sequence of Sema4A, RT-PCR was performed using a sense 5′-AGACTGGCCTCTTACCACTGGAGTCATG-3′ (SEQ ID NO:10) oligonucleotide primer and an antisense 5′-TAGTTGTCGGCATCTACGTCACTG-3′ (SEQ ID NO:11) oligonucleotide primer (94° C. for 30 sec; 60° C. for 30 sec; 72° C. for 30 sec; 30 cycles).

Production of a Soluble Sema4A Protein

A truncated form of Sema4A cDNA was prepared from the full-length Sema4A cDNA by PCR using a pair of oligonucleotide primers containing a sense sequence including a SalI site 5′-AGGTCGACCCATCTGGTGACCATCTCAGGCTGACCATGGC-3′ (SEQ ID NO:12) and an antisense sequence including a BglII site 5′-ATAGATCTGTACTTACTTTGGGCAGCCATGGAAGCTCCGC-3′ (SEQ ID NO:13). The resulting SalI-BglII fragments were used to replace the SalI-BamHI DNA fragments of the pEFBos human IgG1 Fc cassette. To produce a Sema4A-Fc protein, stable P3U1 plasmacytoma transfectants carrying the expression plasmid were established by electroporation. Briefly, aliquots of 10⁷ cells were transfected with 50 μg of the plasmid DNA digested with HindIII and 5 μg of pMC1neo vector digested with BamHI by electroporation. After selection in RPMI medium containing 10% FCS and 0.3 mg/ml of G418 for 10 days, individual G418-resistant colonies were isolated and cloned. The Sema4A-Fc protein was purified from culture supernatants by protein A-Sepharose (Amersham Pharmacia, Piscataway, N.J.).

Transfectants

Sema4A-CHO were generated by introducing full-length FLAG-tagged Sema4A cDNAs in the pEFBos vector and the pMC1neo vector using LIPOFECTAMINE™ Plus 2000 (Life Technologies, Carlsbad, Calif.), a cationic lipid formulation for transfection. Sema4A-CHO were selected by anti-FLAG mAb (M2, Sigma, St. Louis, Mo.) and cloned. As a control transfectant, CHOneo was generated by transfection of CHO cells with the pMC1 neo vector alone.

Anti-Sema4A mAb

Anti-Sema4A (SK31, rat IgG2a) was established as follows. Rats were immunized three times and boosted once wiTh 100 mg of Sema4A-Fc protein. Rat splenocytes were fused with P3U1 cells, and 7 days later, hybridomas were tested for the production of specific antibodies using Sema4A-CHO by flow cytometry.

Flow Cytometric Analysis for Expression of Sema4A and its Counter-Receptor

Anti-Sema4A, Sema4A-Fc and isotype-matched control Igs were biotinylated using a biotinylation kit (Boehringer Mannheim). For flow cytometric analysis of Sema4A or its counter-receptor, aliquots of 10⁶ cells were incubated with biotinylated anti-Sema4A, Sema4A-Fc, or control Igs on ice for 1 hr containing 5 mg/ml of Fc block (PharMingen, BD Biosciences, Palo Alto, Calif.). After washing with staining buffer, the cells were stained for 20 min with FITC-conjugated streptavidin (PharMingen, BD Biosciences, Palo Alto, Calif.). Cells were then washed and analyzed by a flow cytometer.

In vitro T cell Stimulation

For T cell proliferation assays, CD4+ T cells were prepared from splenocytes using Magnetic Cell Sorting (MACS) (Miltenyi Biotech, Germany). Cells (1×10⁵) were stimulated with 5 μg/ml of anti-CD3 (2C11; PharMingen, BD Biosciences, Palo Alto, Calif.) coated flat-bottomed 96-well plates in the absence or presence of anti-CD28 (10 μg/ml) for 48 hr. For T cell differentiation analysis, naïve CD4+ T cells were stimulated with anti-CD3 plus anti-CD28 in the absence or presence of Sema4A-Fc for 6 days, which was supplemented with IL-12 plus anti-IL-4 to generate Th1-like cells or with IL-4 to generate Th2-like cells. Then, the harvested cells were re-stimulated with anti-CD3 plus anti-CD28 for 48 hr. For MLRs, DCs were generated from the bone marrow progenitors of C57BL/6 mice, using GM-CSF, as previously described. Irradiated (3000 rad) DCs from C57BL/6 mice were cultured with CD4+ T cells (5×10⁴ cells/well) derived from BALB/c mice with or without Sema4A-Fc or human IgG1 (PharMingen, BD Biosciences, Palo Alto, Calif.) in flat-bottomed 96-well plates for 72 hr. Cells were pulsed wiTh2 mCi of [³H]thymidine for the final 12 hr of incubation. For IL-2 production, the levels of IL-2 in the culture supernatants were measured using an ELISA kit (Endogen, Perbio Science AB, Bonn, Germany). For the MLR using fixed fully maturated DCs, bone marrow-derived DCs of C57BL/6 mice were treated with anti-CD40 (5 mg/ml) for 24 hr, then fixed with 0.8% paraformaldehyde and used as stimulators.

B Cell Proliferation Assay

Nonadherent splenic B cells from C57BL/6 mice (6-8 weeks) were isolated with a combination of anti-Thy1.2 (F7D5, Seroteck Ltd, U.K.) and rabbit complement (Wako, Japan). The remaining B cells were further fractionated through a Percoll gradient of 50%, 60%, 66%, and 70%, and the cells at the interface between 66% and 70% layers were collected. The resulting small resting B cells (1×10⁵ cells/well) were cultured with or without 1 mg/ml of anti-CD40 (HM40-3, PharMingen, BD Biosciences, Palo Alto, Calif.) and 10 U/ml of IL-4 (Genzyme, Cambridge, Mass.) in the presence of either Sema4A-Fc or CD100-Fc in flat-bottomed 96-well plates for 72 hr. Cells were pulsed wiTh2 mCi of [³H]thymidine for the last 16 hr.

IL-12 Production Assay

IL-12 was quantitated after culturing DCs (1×10⁶ cells/well) in 24-well plates for 72 hr with or without anti-CD40 (5 mg/ml) plus either Sema4A-Fc or CD100-Fc. The mature IL-12p70 heterodimer was detected using a mouse IL-12 ELISA kit (Amersham Pharmacia, Piscataway, N.J.).

In vivo T Cell Priming

Eight-week-old C57BL/6 mice were immunized wiTh 10 mg of KLH in CFA (Sigma, St. Louis, Mo.) into the hind footpads. Either Sema4A-Fc (100 mg/mouse/day) or human IgG1 (50 mg/mouse/day) was injected intravenously for 4 days after immunization. Five days after the immunization, CD4+ T cells were purified from the draining lymph nodes by MACS and 1×10⁵ cells were stimulated with various concentrations of KLH in the presence of irradiated (3000 rad) splenocytes (1×10⁶ cells/well) of C57BL/6 mice in flat-bottomed 96-well plates for 72 hr. For proliferation assays, cells were pulsed wiTh2 mCi of [³H]thymidine for the last 16 hr. Levels of IL-4 and IFN-gamma in the culture supernatants were measured by ELISA (R&D Systems, Minneapolis, Minn.).

Induction and Blocking of EAE

EAE was induced in 8 to 12 week-old C57BL/6 mice by subcutaneous injections of 100 mg/ml of mouse/rat MOG-peptide (MEVGWYRSPFSRVVHLYRNGK (SEQ ID NO:14), Kurabo, Japan) in CFA including heat inactivated Mycobacterium tuberculosis into the femoral region on both sides. Additionally, pertussis toxin (100 ng; List Biological Labs, Campbell, Calif.) was intravenously injected on days 0 and 2. Either anti-Sema4A or rat IgGs (ICN Pharmaceuticals, Inc., Costa Mesa, Calif.) (100 mg/mouse/day each) was injected intraperitoneally daily for five days after the immunization. Animals were observed daily, and neurological defects were quantified on an arbitrary clinical scale. The in vitro responses of MOG-specific T cells were determined as follows: seven days after the immunization with the same procedure, CD4+ T cells were purified from the draining lymph nodes by MACS and 1×10⁵ cells were stimulated with various concentrations of MOG-peptide in the presence of irradiated (3000 rad) splenocytes (1×10⁶ cells/well) of C57BL/6 mice in flat-bottomed 96-well plates for 72 hr. For proliferation assay, cells were pulsed with Th2 mCi of [³H]thymidine for the last 16 hr. Levels of IL-4 and IFN-γ in the culture supernatants were measured using an ELISA kit (R&D Systems, Minneapolis, Minn.).

Construction of the cDNA Library and Expression Cloning

Total cellular RNA was isolated from EL-4 cells by guanidinium isothiocyanate gradient centrifugation, and mRNA was selected using oligo(dT)-coupled magnetic beads (PolyA Tract mRNA Isolation System, Promega). Double-stranded cDNA primed with oligo(dT) was synthesized using a SUPERSCRIPT™ cDNA synthesis kit (Invitrogen, Carlsbad, Calif.). A BstXI adaptor (Invitrogen, Carlsbad, Calif.) was added to the cDNA, which was size-fractionated by electrophoresis on a 1% agarose gel. cDNAs larger than 800 bp were recovered and ligated to BstXI-digested pME18S vector. E. coli DH10B cells (Invitrogen, Carlsbad, Calif.) were transformed with the ligated DNA by electroporation. Aliquots of 2.0×10⁷ independent clones were used to transfect COS7 cells. COS7 cells were transfected with plasmid DNAs using LIPOFECTAMINE™ PLUS™ (Invitrogen, Carlsbad, Calif.). Three days after transfection, the cells were harvested, re-suspended to a concentration of 5×10⁶ cells/ml in PBS containing 5% FCS, 2.5 μg/ml of Fc block (PharMingen, BD Biosciences, Palo Alto, Calif.) and 5 μg/ml of biotinylated Sema4A-Fc or biotinylated human immunoglobulin Fc, and incubated on ice for 1 hr. The cells were washed with ice-cold PBS and suspended to 5×10⁶ cells/ml in PBS containing Dynabeads M-280 streptavidin (Dynal A.S., Oslo, Norway). After incubation for 30 min, the cells were washed with ice-cold PBS ten times using a Magnetic Particle Concentrator (Dynal A.S., Oslo, Norway). The extrachromosomal plasmid DNA was extracted from binding cells by the Hirt method. The plasmid DNA was introduced into E. coli DH10B cells by electroporation, then applied to the second and third transfection by protoplast fusion. Magnetic sorting was repeated three times as described above.

While the invention has been described with respect to preferred embodiments, those skilled in the art will readily appreciate that various changes and/or modifications can be made to the invention without departing from the spirit or scope of the invention as defined by the appended claims. All documents cited herein are incorporated by reference in their entireties.

What is claimed is:

1. An antibody or antibody derivative, wherein said antibody or antibody derivative specifically binds to human Sema4A protein.

2. The antibody or antibody derivative according to claim 1, wherein said antibody or antibody derivative inhibits the costimulatory effect of human Sema4A protein on T cell activation.

3. A pharmaceutical composition comprising an antibody or antibody derivative according to claim 1 and a component selected from the group consisting of: an excipient, an adjuvant, a diluent and a carrier.

4. A pharmaceutical composition comprising an antibody or antibody derivative according to claim 2 and a component selected from the group consisting of: an excipient, an adjuvant, a diluent and a carrier.
